SEQUENCE LISTING (17 sequences)

SEQ ID NO: 1 - RNA, 19 nt, artificial
  RNAi target sequence specific for claudin 1
gcaaagcaccgggcagaua

SEQ ID NO: 2 - RNA, 21 nt, artificial
  RNAi sense sequence specific for claudin 1
auagacgggccacgaaacguu

SEQ ID NO: 3 - RNA, 21 nt, artificial
  RNAi anti-sense strand specific for claudin 1
cguuucguggcccgucuauuu

SEQ ID NO: 4 - RNA, 19 nt, artificial
  RNAi target sequence specific for claudin 1
gaacaguacuuugcaggca

SEQ ID NO: 5 - RNA, 21 nt, artificial
  RNAi sense sequence specific for claudin 1
acggacguuucaugacaaguu

SEQ ID NO: 6 - RNA, 21 nt, artificial
  RNAi anti-sense strand specific for claudin 1
cuugucaugaaacguccguuu

SEQ ID NO: 7 - RNA, 19 nt, artificial
  RNAi target sequence specific for claudin 1
uuucaggucuggcgacauu

SEQ ID NO: 8 - RNA, 21 nt, artificial
  RNAi sense sequence specific for claudin 1
uuacagcggucuggacuuuuu

SEQ ID NO: 9 - RNA, 21 nt, artificial
  RNAi anti-sense strand specific for claudin 1
aaaguccagaccgcuguaauu

SEQ ID NO: 10 - DNA, 21 bp, Artificial
  claudin-1 specific RNAi sense strand
uacauaggcauaguucaugnn

SEQ ID NO: 11 - DNA, 21 bp, Artificial
  claudin-1 specific RNAi sense strand
caugaacuaugccuauguann

SEQ ID NO: 12 - DNA, 21 bp, Artificial
  claudin-1 specific RNAi sense strand
aacguaugcaguuaauuccnn

SEQ ID NO: 13 - DNA, 21 bp, Artificial
  claudin-1 specific RNAi sense strand
ggaauuaacugcauacguunn

SEQ ID NO: 14 - DNA, 21 bp, Artificial
  claudin-1 specific RNAi sense strand
ugaagaucuauguauguggnn

SEQ ID NO: 15 - DNA, 21 bp, Artificial
  claudin-1 specific RNAi sense strand
ccacauacauagaucuucann

SEQ ID NO: 16 - protein, 28 aa, Artificial
  soluble CLDN-1 peptide fragment consensus sequence
SCVSQSTGQXQCKVFDSLLNLSSTLQAT

SEQ ID NO: 17 - protein, 49 aa, artificial
  soluble CLDN-23 peptide fragment
PGWRLVKGFLNQPVDVELYQGLWDMCREQSSRERECGQTDQWGYFEAQP

(19) United States
(12) Patent Application Publication
(10) Pub. No.: US 2016/0000777 A1
(43) Pub. Date: Jan. 7, 2016

(54) METHODS OF USING HISTAMINE RECEPTOR AGONISTS AND ANTAGONISTS

(71) Applicant: UNIVERSITY OF ROCHESTER, Rochester, NY (US)

(72) Inventors: Lisa A. BECK, Rochester, NY (US); Anna DE BENEDETTO, Rochester, NY (US); Takeshi YOSHIDA, Rochester, NY (US)

(73) Assignee: UNIVERSITY OF ROCHESTER, Rochester, NY (US)

(21) Appl. No.: 14/769,549

(22) PCT Filed: Feb. 21, 2014
(86) PCT No.: PCT/US2014/017556

§ 371 Date: Aug. 21, 2015

Related U.S. Application Data
(60) Provisional application No. 61/767,512, filed on Feb. 21, 2013.

(51) Int. Cl. A61K 31/496; A61K 9/00; A61K 31/417; A61K 45/06; A61K 31/4164; A61K 31/495

(52) U.S. Cl. CPC A61K 31/496; A61K 31/4164; A61K 31/495; A61K 31/417; A61K 45/06; A61K 9/0014

(57) ABSTRACT

This invention relates to a transdermal drug formulation that includes a pharmaceutically suitable carrier; an effective amount of a therapeutic agent; and a histamine type 4 receptor (“H4R”) agonist, as well as a transdermal vaccine formulation that includes a pharmaceutically suitable carrier; an effective amount of an antigen or antigen-encoding nucleic acid molecule present in the carrier, and optionally one or more adjuvants; and an H4R agonist. The present invention also relates to transdermal delivery device including such formulations and methods of administering such formulations. The present invention also relates to methods of enhancing epithelial barrier formation in a patient involving administering to the patient at a site of epithelial disruption an amount of a formulation that comprises an H4R antagonist. The present invention also relates to a method of inhibiting pathogen infection or local spread of infection in the epithelia using an H4R antagonist, an H1R antagonist, or a combination thereof.

This application claims benefit of U.S. Provisional Patent Application Ser. No. 61/767,512, filed Feb. 21, 2013, which is hereby incorporated by reference in its entirety.

This invention was made with support from the National Institutes of Health under grant AR007472-21. The U.S. government has certain rights in this invention.

### FIELD OF THE INVENTION

This invention relates to formulations including histamine receptor agonists, formulations including histamine receptor antagonists, and methods of using such formulations.

### BACKGROUND OF THE INVENTION

Atopic dermatitis (“AD”) is the most common chronic inflammatory skin disease and is characterized by cutaneous hyperreactivity to allergens and propensity for microbial colonization and infection. Despite its high prevalence, effects on quality-of-life and economic burden, there are still few effective treatments for AD, and most have focused on inhibiting inflammation.

For a long time, AD treatments have targeted inflammation. Based on important discoveries over the last decade, it is thought that AD develops in part as the consequence of an acquired or genetic defect of the skin's barrier. These defects enable microbes, allergens, antigens, and irritants to be sensed by the skin, causing a vigorous immune response that is a key driver of the cutaneous inflammation observed in skin lesions. That notion opens new opportunities for therapeutic intervention or prevention by harnessing drugs that could enhance or repair skin barrier function rather than just suppress the inflammatory response as either a preventative approach or as a therapeutic Rx. It is widely accepted that the stratum corneum (“SC”) is dysfunctional in AD as the result of a number of abnormalities including but not limited to reduced lipids, abnormal epidermal differentiation, loss of function filaggrin mutations, or simply the disruption that occurs from the intense itch-scratch cycle that characterizes this disease. But the epidermis has an additional barrier structure called tight junctions (“TJ”), found just below the SC. In epithelial cells, TJs function as the “gate” for paracellular (i.e., space between adjacent cells) passage of ions and solutes, which affects water transport.

Histamine has long been recognized as a potent inducer of pruritus, and it is highly expressed in the skin of subjects with AD, acute and chronic urticaria, as well as other conditions characterized by mast cell activation. Histamine is also an important mediator in allergic diseases that affect other organs (e.g., asthma, food allergy, allergic rhinitis, allergic conjunctivitis, etc). Histamine can bind to four receptors (H1R, H2R, H3R and H4R). Keratinocytes have been shown to express H1R and H2R, and very recently H4R.

Many of the allergic and inflammatory actions of histamines are thought to be mediated by H1R, a Gαq/11 receptor. FDA-approved antihistamines block either H1R or H2R or both; relatively selective H3R and H4R blockers are currently in various stages of development by many pharma/biotech companies. Recently published papers have highlighted the role of histamine (and mostly H1R) in epidermal barrier (Gschwandtner et al., “Histamine Suppresses Epidermal Keratinocyte Differentiation and Impairs Skin Barrier Function in a Human Skin Model,” Allergy 68:37-47 (2013); Lin et al., Topical Antihistamines Display Potent Anti-Inflammatory Activity Linked in Part to Enhanced Permeability Barrier Function,” Journal of Investigative Dermatology 133:469-478 (2013)). Briefly, it is has been shown in a murine model that H1R and H2R antagonists improved skin barrier function, mainly after acute barrier disruption induced by tape stripping (Lin et al., Topical Antihistamines Display Potent Anti-Inflammatory Activity Linked in Part to Enhanced Permeability Barrier Function,” Journal of Investigative Dermatology 133:469-478 (2013), which is hereby incorporated by reference in its entirety). The barrier recovery was associated with enhanced expression of markers of keratinocyte terminal differentiation (e.g., filaggrin, loricirn) as well as lipids (Lin et al., Topical Antihistamines Display Potent Anti-Inflammatory Activity Linked in Part to Enhanced Permeability Barrier Function,” Journal of Investigative Dermatology 133:469-478 (2013)). Consistent with this observation, Gschwandtner et al. (“Histamine Suppresses Epidermal Keratinocyte Differentiation and Impairs Skin Barrier Function in a Human Skin Model,” Allergy 68:37-47 (2013)) demonstrated in human keratinocytes that histamine reduced the expression of stratum corneum and granulosum barrier proteins (e.g. filaggrin, Loricrin, claudin1, occludin) and this was selectively blocked by a H1R antagonist.

There are no FDA-approved treatments to repair skin barrier impairment in AD as well as other inflammatory skin disease (e.g., psoriasis, contact dermatitis, urticarial (hives) wound healing, UV or ionizing radiation damage) or epithelial barrier in diseases or disorders involving other organs such as gastrointestine (e.g., IBD, Coeliac disease, infectious colitis etc.) or respiratory system (e.g., Asthma, Allergic rhinitis, sinusitis, etc). Currently approved and off-label therapies used to treat AD focus on dampening the inflammatory response.

The present invention is directed to overcoming these and other limitations in the art.

### SUMMARY OF THE INVENTION

A first aspect of the present invention relates to a transdermal drug formulation that includes a pharmaceutically suitable carrier; an effective amount of a therapeutic agent; and a histamine type 4 receptor (“H4R”) agonist.

A second aspect of the present invention relates to a transdermal vaccine formulation that includes a pharmaceutically suitable carrier; an effective amount of an antigen or antigen-encoding nucleic acid molecule present in the carrier, and optionally one or more adjuvants; and an H4R agonist.

A third aspect of the present invention relates to a transdermal delivery device that includes a transdermal drug formulation or a transdermal vaccine formulation of according to the present invention.

A fourth aspect of the present invention relates to a method of disrupting an epithelial barrier. The method involves administering to an epithelial site an amount of an H4R agonist that transiently disrupts tight junctions, thereby disrupting barrier formation at the epithelial site.

A fifth aspect of the present invention relates to a method of administering a transdermal drug formulation to a subject. The method involves applying a transdermal drug formulation of the present invention to an epithelial site on the subject.

A sixth aspect of the present invention relates to a method of administering a transdermal vaccine formulation to a subject. The method involves applying a transdermal vaccine formulation of the present invention to an epithelial site on the subject.

A seventh aspect of the present invention relates to a method of enhancing epithelial barrier formation in a patient. The method involves administering to the patient at a site of epithelial disruption an amount of a formulation that comprises an H4R antagonist, thereby enhancing barrier formation at the site.

An eighth aspect of the present invention relates to a method of promoting epithelial function in an individual having compromised or immature epithelial function. The method providing a formulation comprising H4R antagonist, an H1R antagonist, or a combination thereof that enhances tight junction formation between epithelial cells; and administering the formulation to a region of epithelia on an individual having reduced epithelial function at the region, thereby enhancing tight junction formation between epithelial cells and promoting epithelial function in the individual.

A ninth aspect of the present invention relates to a method of inhibiting pathogen infection or local spread of infection in the epithelia. The method involves providing a formulation comprising an H4R antagonist, an H1R antagonist, or a combination thereof; and applying to a region of epithelia on an individual that is susceptible to pathogen infection an amount of the formulation that is effective to enhance epithelial barrier formation at the application site, thereby rendering the application site less susceptible to pathogen infection or local spread of infection.

It has recently been demonstrated by the inventors that a TJ defect exists in the epidermis of AD subjects, with reduced expression of claudin-1, -4 and -23, in association with a remarkable impairment of TJ barrier function. Histamine, as well other amines such as thrombin, disrupt TJ in vascular and corneal endothelial cells by inducing myosin light chain (MLC) phosphorylation. Histamine has also been shown to reduce the expression of a key TJ molecule, ZO-1 in nasal and retinal epithelial cells, and this effect was at least partially abrogated by pretreatment with the H1R antagonist, mepyriamine, but not by the H2R antagonist, ranitidine. Histamine was also found to enhance TJ paracellular permeability in airway epithelial cells.

Studies by the inventors, as described herein, have also shown that histamine significantly reduced TJ integrity between epithelial cells such as keratinocytes, and this effect was blocked by a H4R antagonist. This data provides evidence that histamine and H4R agonists can temporarily induce TJ disruption for the purposes of drug delivery or as a vaccination strategy. Selective H4R agonists can be used to open TJ, inducing a temporary barrier defect when needed (e.g. transepidermal or mucosal drug delivery, transcutaneous vaccine). Alternatively, blockade of histamine induced barrier disruption can be used to treat disease conditions or disorders where histamine and/or other H4R endogenous ligands otherwise promote barrier disruption.

The invention can also be applied to the H4R downstream signaling pathway(s) to identify other targets responsible for the modulation of TJ composition and/or function; endogenous and exogenous H4R ligands as well as additional selective antagonists; and the effect of other histamine-like amines and receptors on epidermal barrier function in keratinocytes but also other epithelial cells (e.g. nasal, lung, bladder and intestinal).

### BRIEF DESCRIPTION OF THE DRAWINGS

FIGS. 1A-D are bottom perspective (FIG. 1A) and cross sectional (FIGS. 1B, 1C, and 1D) views of one embodiment of a transdermal patch according to the present invention.

FIGS. 2A-2C are graphs showing results that demonstrate, in cultured primary human keratinocytes (PHK), histamine reduced TJ integrity. Histamine dose-dependently reduced (FIG. 2A) transepithelial electric resistance (TEER) and it (FIG. 2B) enhanced Permeability to Fluorescein. TEER is shown as Mean Area under the curve (AUC) ±SEM on n=10 experiments; permeability is shown as Mean Fold of control (FOC) ±SEM of n=9. *p<0.05; **p<0.001, ***p<0.0001. FIG. 2C also shows results demonstrating that in PHK, histamine significantly reduced TEER peak. PHK were differentiated in DMEM serum free media ±histamine (100 μM). TEER is shown as Mean±SEM of n=4 experiments. **p<0.01.

FIGS. 3A and 3B are bar graphs showing results that demonstrate, in an ex-vivo model, using skin explanted and the adapted microsnap-well system, histamine (100 μM) (FIG. 3A) reduced TEER (0.7 fold) and (FIG. 3B) enhanced Fluorescein permeability flux (20 minutes time point; 1.3 fold). TEER is shown as Mean Fold of control (FOC) ±SEM on n=3; permeability is shown as Fold of control (FOC) Mean±SEM of n=3. *p<0.05; **p<0.001, ***p<0.0001.

FIGS. 4A and 4B are bar graphs showing results that demonstrate, in cultured primary human keratinocytes, Cetirizine (Cet, 10 μM—H1R antagonist) blocks histamine-mediated (FIG. 4A) TEER reduction (n=4) and (FIG. 4B) permeability enhancement (n=9). TEER is shown as Mean Area under the curve (AUC) ±SEM; permeability is shown as Mean Fold of control (FOC) ±SEM. *p<0.05; **p<0.001, ***p<0.0001.

FIGS. 5A-5C are graphs showing results that demonstrate, in cultured primary human keratinocytes, JNJ7777120 (JNJ, 10 μM—H4R antagonist) blocks histamine-mediated (FIG. 5A) TEER reduction (n=4) and (FIG. 5B) permeability enhancement (n=9). TEER is shown as Mean Area under the curve (AUC) ±SEM; permeability is shown as Mean Fold of control (FOC) ±SEM. *p<0.05; **p<0.001, ***p<0.0001. FIG. 5C also shows results demonstrating that, in PHK, JNJ7777120 (JNJ, 10 μM—H4R antagonist) blocks histamine-mediate TEER reduction. TEER is shown as Mean±SEM of n=5; **p<0.001.

FIG. 6 is an image of a Western blot showing results demonstrating that the H1R antagonist, Cetirizine, blocks the effect of histamine on pro-Filaggrin (pro-FLG) expression. A representative Western blot is shown (n=4). Pro-FLG expression in PHK at day 5 post differentiation in media control is shown in lane 1 and, as shown in lane 2, its expression was significantly reduced in PHK treated with 100 μM histamine. This reduction of pro-FLG expression was prevented by co-treating PHK with 100 μM histamine and 10 μM Cetirizine (lane 3 and 4).

FIGS. 7A and 7B are graphs showing results demonstrating that H1R expression is reduced in skin of AD subjects. Expression of H1R (HRH1) (FIG. 7A) and H4R (HRH4) (FIG. 7B) was evaluated by qPCR in skin biopsies of AD (n=6-8; AD_NL: non-lesional and AD_L: lesional) and non-atopic control (n=10; NA). Expression was normalized to GAPDH gene. RVU: relative value unit. *p<0.05; **p<0.01.

### DETAILED DESCRIPTION OF THE INVENTION

The present invention relates to, inter alia, formulations including a histamine type 4 receptor (“H4R”) agonist, as well as the use of H4R agonists to disrupt the epithelial barrier. Several other aspects of the invention relate to formulations including H4R antagonists, H1R antagonists, or a combination thereof, the treatment of diseases or disorders associated with epithelial barrier disruption, and the use of such formulations to promote or enhance epithelial barrier function.

One aspect of the present invention relates to a transdermal drug formulation that includes a pharmaceutically suitable carrier; an effective amount of a therapeutic agent; and an H4R agonist. Another aspect of the present invention relates to a transdermal vaccine formulation that includes a pharmaceutically suitable carrier; an effective amount of an antigen or antigen-encoding nucleic acid molecule present in the carrier, and optionally one or more adjuvants; and an H4R agonist.

H4R agonists that are suitable for use in accordance with the present invention include, but are not limited to, endogenous H4R ligands, histamine and histamine analogs, and combinations thereof. Exemplary H4R agonists include, without limitation, histamine, liver-expressed chemokine (“LEC”)/CCL16, 4-methylhistamine, VUF-8430 (2-[(Aminoiminomethyl)amino]ethyl carbamimidothioic acid ester), OUP-16, and combinations thereof (Nakayama et al., “Liver-Expressed Chemokine/CC Chemokine Ligand 16 Attracts Eosinophils by Interacting with Histamine H4 Receptor,” J. Immunol. 173(3): 2078-83 (2004) and Lim et al., “Evaluation of Histamine H₁-, H₂-, and H₃-Receptor Ligands at the Human Histamine H₄ Receptor: Identification of 4-Methylhistamine as the First Potent and Selective H₄ Receptor Agonist,”JPET 314(3): 1310-1321 (2005), each of which is hereby incorporated by reference in its entirety). In certain embodiments, the H4R agonist may be an H4R-specific agonist.

The transdermal drug or vaccine formulations according to the present invention may include one or more H4R agonists. In one embodiment, the transdermal drug or vaccine formulation includes a second H4R agonist that is different from the first.

The transdermal drug or vaccine formulations according to the present invention may also include a histamine type 1 receptor (“H1R”) agonist.

Exemplary H1R agonists include, but are not limited to, histamine, 2-Methylhistamine, 2-(3-Bromophenyl)histamine, Histaprodifen, 8R-Lisuride, 2-(2-thiazolyl)ethylamine (“TEA”), 2-pyridylethylamine (“PEA”), 2-pyridylethylamine (PEA) dihydrochloride, and combinations thereof (Lim et al., “Evaluation of Histamine H₁-, H₂-, and H₃-Receptor Ligands at the Human Histamine H₄ Receptor: Identification of 4-Methylhistamine as the First Potent and Selective H₄ Receptor Agonist,”JPET 314(3): 1310-1321 (2005), which is hereby incorporated by reference in its entirety). In certain embodiments, the H1R agonist is an H1R-specific agonist.

The transdermal drug or vaccine formulations according to the present invention may include one or more H1R agonists. In one embodiment, the transdermal drug formulation includes a second H1R agonist that is different from the first.

The transdermal drug or vaccine formulations according to the present invention may include a single histamine receptor agonist that agonizes or activates more than one histamine receptor. For example, the transdermal drug or vaccine formulations may include a single agent that is an agonist to both H1R and H4R. In other embodiments, the formulations include multiple agents. For example, the formulation may include an agent that is an H1R agonist and an agent that is an H4R agonist.

In one embodiment, the transdermal drug or vaccine formulation does not include an H2R agonist. In one embodiment, the transdermal drug or vaccine formulation does not include histamine.

The transdermal drug or vaccine formulations according to the present invention may also include one or more additional agents that disrupt tight junctions. For instance, the transdermal drug or vaccine formulations may include an agent that transiently disrupts a claudin protein function within tight junctions. For instance, the transdermal drug or vaccine formulations may include an agent that transiently disrupts claudin-1, -4, and/or -23 function within tight junctions. In one embodiment, the transdermal drug or vaccine formulation includes an H1R agonist, a second H4R agonist, an agent that transiently disrupts claudin-1, -4, and/or -23 function within tight junctions, or a combination thereof. Exemplary agents that transiently disrupt claudin-1 and/or -23 function are described in U.S. Patent App. Pub. Nos. 2013/0046257 and 2013/0045267 to Beck et al., which are hereby incorporated by reference in their entirety.

Agents that can decrease claudin-1, -4, and/or -23 expression include, without limitation, antisense nucleic acid molecules, including interfering RNA molecules (RNAi), certain interleukins, and fatty acid agents.

An important feature of RNAi affected by siRNA is the double stranded nature of the RNA and the absence of large overhanging pieces of single stranded RNA, although dsRNA with small overhangs and with intervening loops of RNA has been shown to effect suppression of a target gene. In this specification, it will be understood that in this specification the terms siRNA and RNAi are interchangeable. Furthermore, as is well-known in this field, RNAi technology may be carried out by siRNA, miRNA or shRNA or other RNAi inducing agents. Although siRNA will be referred to in general in the specification. It will be understood that any other RNA inducing agent may be used, including shRNA, miRNA or an RNAi-inducing vector whose presence within a cell results in production of an siRNA or shRNA targeted to a target claudin-1, -4, and/or -23 transcript.

RNA interference is a multistep process and is generally activated by double-stranded RNA (dsRNA) that is homologous in sequence to the targeted claudin-1, -4, and/or -23 gene. Introduction of long dsRNA into the cells of organisms leads to the sequence-specific degradation of homologous gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted claudin-1, -4, and/or -23 RNA molecule. The endonuclease activity hydrolyzes the claudin-1 RNA at the site where the antisense strand is bound. Therefore, RNAi is an antisense mechanism of action, as a single stranded (ssRNA) RNA molecule binds to the target claudin-1, -4, and/or -23 RNA molecule and recruits a ribonuclease that degrades the claudin-1, -4, and/or -23 RNA.

An “RNAi-inducing agent” or “RNAi molecule” is used in the invention and includes for example, siRNA, miRNA or shRNA targeted to a claudin-1, -4, and/or -23 transcript or an RNAi-inducing vector whose presence within a cell results in production of an siRNA or shRNA targeted to a target transcript. Such siRNA or shRNA comprises a portion of RNA that is complementary to a region of the target claudin-1, -4, and/or -23 transcript. Essentially, the “RNAi-inducing agent” or “RNAi molecule” downregulates expression of the targeted claudin-1, -4, and/or -23 protein via RNA interference.

Preferably, siRNA, miRNA or shRNA targeting claudin-1, -4, and/or -23 proteins are used.

Exemplary RNAi specific for claudin-23 is available from Santa Cruz Biotechnology (products sc-77716 and sc-77716-SH), as well as Applied Biosystems (products s44021, s-44022, s-44023, 128551, 128552, 290262, and 284899), which are hereby incorporated by reference in their entireties.

Exemplary RNAi specific for claudin-4 may be found in WO/2005/058362, which is hereby incorporated by reference in its entirety.

Exemplary RNAi specific for claudin-1 are listed below:

(SEQ ID NO: 1)

| | |
|---|---|
| CLDN1 (1) target sequence: | GCAAAGCACCGGGCAGAUA |

(SEQ ID NO: 2)

| | |
|---|---|
| Sense sequence: | AUAGACGGGCCACGAAACGUU |

(SEQ ID NO: 3)

| | |
|---|---|
| Anti-sense strand: | CGUUUCGUGGCCCGUCUAUUU |

(SEQ ID NO: 4)

| | |
|---|---|
| CLDN1 (2) target sequence: | GAACAGUACUUUGCAGGCA |

(SEQ ID NO: 5)

| | |
|---|---|
| Sense strand: | ACGGACGUUUCAUGACAAGUU |

(SEQ ID NO: 6)

| | |
|---|---|
| Anti-sense strand: | CUUGUCAUGAAACGUCCGUUU |

(SEQ ID NO: 7)

| | |
|---|---|
| CLDN1 (3) target sequence: | UUUCAGGUCUGGCGACAUU |

(SEQ ID NO: 8)

| | |
|---|---|
| Sense sequence: | UUACAGCGGUCUGGACUUUUU |

(SEQ ID NO: 9)

| | |
|---|---|
| Anti-sense strand: | AAAGUCCAGACCGCUGUAAUU |

Another exemplary RNAi product specific for claudin-1 includes the mixture of the following dsRNA (A+B+C):

Sense Strand (A):
(SEQ ID NO: 10)
UACAUAGGCAUAGUUCAUGtt
(SEQ ID NO: 11)
CAUGAACUAUGCCUAUGUAtt
Sense Strand (B):
(SEQ ID NO: 12)
AACGUAUGCAGUUAAUUCCtt
(SEQ ID NO: 13)
GGAAUUAACUGCAUACGUUtt
Sense Strand (C):
(SEQ ID NO: 14)
UGAAGAUCUAUGUAUGUGGtt
(SEQ ID NO: 15)
CCACAUACAUAGAUCUUCAtt

Other agents that can be used to interrupt claudin-1, -4, and/or -23 activity include soluble fragments of claudin-1, -4, and/or -23 that consist essentially of one or more extracellular domains of claudin-1, -4, and/or -23, which when delivered to epithelial cells (e.g., keratinocytes) (subsequent to TJ disruption within a region or prior to TJ formation in a particular region) can inhibit claudin-1, -4, and/or -23 dimerization and thereby reduce the efficacy of TJ formation. By way of example, a 27-amino acid peptide corresponding to a portion of the first EL domain (Cldn-153-80) has been shown reversibly to interfered with epithelial barrier function by inducing the rearrangement of key TJ proteins: occludin, claudin-1, junctional adhesion molecule-A, and zonula occludens-1 (Mrsny et al. “A Key Claudin Extracellular Loop Domain Is Critical for Epithelial Barrier Integrity,” Am. J. Pathol. 172(4):905-915 (2008), which is hereby incorporated by reference in its entirety). A soluble (human) CLDN-1 peptide comprises the consensus amino acid sequence of SEQ ID NO: 16 as follows:

SCVSQSTGQ[I/V]QCKVFDSLLNLSSTLQAT

By way of an additional example, a soluble fragment of the first extracellular loop of claudin-23 (Genbank Accession NP_{—}919260, which is hereby incorporated by reference in its entirety) may be used. In one embodiment, a soluble (human) CLDN-23 peptide fragment is derived from the amino acid sequence of SEQ ID NO: 17 as follows:

PGWRLVKGFLNQPVDVELYQGLWDMCREQSSRERECGQTDQWGYFEAQP

Homologs of these sequences can also be identified based on conserved amino acid substitutions as is well known in the art.

Other agents include antibodies or aptamers that target the claudin-1, -4, and/or -23 extracellular domains, particularly those that target extracellular loops such as the first EL domain. Antibodies that bind to this region of Claudin-1 are identified in Fofana et al., “Monoclonal Anti-claudin 1 Antibodies Prevent Hepatitis C Virus Infection of Primary Human Hepatocytes,” Gastroenterology 139(3):953-64 (2010), which is hereby incorporated by reference in its entirety.

Suitable fatty acid agents can be identified by screening for displacement of tight junction proteins, including claudin-1, -4, and/or claudin-23, using the procedures identified by Sugibayashia et al., “Displacement of Tight Junction Proteins from Detergent-resistant Membrane Domains by Treatment with Sodium Caprate,” Eur. J. Pharm. Sci. 36(2-3):246-253 (2009); Kurasawa et al., “Regulation of Tight Junction Permeability by Sodium Caprate in Human Keratinocytes and Reconstructed Epidermis,” Biochem Biophys. Res. Commun. 381(2):171-5 (2009), each of which is hereby incorporated by reference in its entirety. One exemplary fatty acid agent is sodium caprate.

Regardless of the embodiment, agents and formulations according to the present invention may be administered via pharmaceutical composition. When administered in the form of a pharmaceutical composition, the composition includes a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active agent(s), together with the adjuvants, carriers and/or excipients. While individual needs may vary, determination of optimal ranges of effective amounts of each component is within the skill of the art. Typical dosages comprise about 0.01 to about 100 mg/kg·body wt. The preferred dosages comprise about 0.1 to about 100 mg/kg·body wt. The most preferred dosages comprise about 1 to about 100 mg/kg·body wt. Treatment regimen for the administration of the agents can also be determined readily by those with ordinary skill in art. That is, the frequency of administration and size of the dose can be established by routine optimization, preferably while minimizing any side effects.

Liposomal or micelle preparations can also be used to deliver the agents of the present invention.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

Like liposomes, micelles have also been used in the art for drug delivery. A number of different micelle formulations have been described in the literature for use in delivery proteins or polypeptides, and others have been described which are suitable for delivery of nucleic acids. Any suitable micelle formulations can be adapted for delivery of the therapeutic protein or polypeptide or nucleic acids of the present invention. Exemplary micelles include without limitation those described, e.g., in U.S. Pat. No. 6,210,717 to Choi et al.; and U.S. Pat. No. 6,835,718 to Kosak, each of which is hereby incorporated by reference in its entirety.

When it is desirable to achieve heterologous expression of a protein that, e.g., promotes claudin-1, -4, and/or claudin-23 expression or RNAi, which, e.g., knocks down claudin-1, -4, and/or claudin-23 expression, then DNA molecules encoding these products can be delivered into the cell. Basically, this includes providing a nucleic acid molecule encoding the desired product, and then introducing the nucleic acid molecule into the cell under conditions effective to express the desired product in the cell. Preferably, this is achieved by inserting the nucleic acid molecule into an expression vector before it is introduced into the cell.

Any suitable viral or infective transformation vector can be used. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, each of which is hereby incorporated by reference in its entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), each of which is hereby incorporated by reference in its entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, each of which is hereby incorporated by reference in its entirety.

Regardless of the type of infective transformation system employed, it should be targeted for delivery of the nucleic acid to a specific cell type. For example, for delivery of the nucleic acid into a cluster of cells, a high titer of the infective transformation system can be introduced directly within the site of those cells so as to enhance the likelihood of cell infection. The infected cells will then express the desired product, in this case RNAi that knocks down expression of claudin-1, -4, and/or claudin-23 or a protein that enhances claudin-1, -4, and/or claudin-23 expression. Alternatively, these infective transformation systems can be administered in combination with a liposomal or micelle preparation, as well as a depot injection.

Ideally, the method involves the local hydrodynamic delivery of the RNAi inducing agent, such as siRNA, miRNA or shRNA etc, to the subject. Although, non-hydrodynamic systemic delivery methods may also be used.

Other delivery methods suitable for the delivery of the RNAi inducing agent (including siRNA, shRNA and miRNA, etc) may also be used. For example, some delivery agents for the RNAi-inducing agents are selected from the following non-limiting group of cationic polymers, modified cationic polymers, peptide molecular transporters, lipids, liposomes and/or non-cationic polymers. Viral vector delivery systems may also be used. For example, an alternative delivery route includes the direct delivery of RNAi inducing agents (including siRNA, shRNA and miRNA) and even anti-sense RNA (asRNA) in gene constructs followed by the transformation of cells with the resulting recombinant DNA molecules. This results in the transcription of the gene constructs encoding the RNAi inducing agent, such as siRNA, shRNA and miRNA, or even asRNA and provides for the transient and stable expression of the RNAi inducing agent in cells and organisms. For example, such an alternative delivery route may involve the use of a lentiviral vector comprising a nucleotide sequence encoding a siRNA (or shRNA) which targets the tight junction proteins. Such a lentiviral vector may be comprised within a viral particle. Adeno-associated viruses (“AAV”) may also be used.

The present invention also includes pharmaceutical or dermatological compositions, which include any of the classes of agents described herein along with an acceptable carrier. The carrier is preferably in the form of a lotion, cream, gel, emulsion, ointment, foam, solution, suspension, mucoadhesive, or paste. The compositions can be applied to a region of skin by spraying or misting a solution or suspension onto the region of skin, or spreading the lotion, cream, gel, emulsion, ointment, foam, mucoadhesive, or paste onto the region of skin. These compositions may also include, e.g., spermicidal agents such as nonoxynol-9, and can be applied externally as well as intravaginally, as needed.

The carrier according to the present invention may include an oil-in-water emulsion. The carrier may also include liposomes or micelles, which are described above. In one embodiment the transdermal drug or vaccine formulation includes a carrier selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

As noted above, the pharmaceutical composition can be a vaccine, preferably a transdermal vaccine formulation that would benefit from TJ disruption at the site of vaccine delivery. The formulation is presented in the transdermal delivery vehicle, as is known in the art. The transdermal vaccine is often presented in the form of a patch worn by the user, whereby moisture from the vaccine recipient's body allows for delivery of the active agents across the skin (i.e., at the site of application).

Any suitable antigen or antigen-encoding nucleic acid molecule, or a combination thereof, can be used in the vaccine formulations of the present invention. Exemplary classes of vaccine antigen include, without limitation, an allergen, an immunogenic subunit derived from a pathogen, a virus-like particle, an attenuated virus particle, or glycoprotein or glycolipid conjugated to an immunogenic polypeptide. Antigen-encoding nucleic acid molecules are also encompassed and can be in the form of naked DNA or expression vectors, as well as infective transformation vectors.

A number of known transdermal vaccine formulations can be modified to include formulations or agents according to the present invention.

One exemplary transdermal vaccine formulation that can be modified is described in U.S. Pat. No. 6,420,176 to Lisziewicz et al., which is hereby incorporated by reference in its entirety. For example, the carrier may comprise one or more of sugar, polylysine, polyethylenimine, polyethylenimine derivatives, and liposomes, together with their derivatives. One preferred carrier of this type is a mannosylated polyethylenimine. The DermaVir transdermal delivery system is believed to employ these types of carriers.

Another exemplary transdermal vaccine formulation that can be modified is described in U.S. Pat. No. 6,869,607 to Buschle et al., which is hereby incorporated by reference in its entirety. For example, the carrier may comprise a solution or emulsion that is substantially free of inorganic salt ions and includes one or more water soluble or water-emulsifiable substances capable of making the vaccine isotonic or hypotonic (e.g., maltose, fructose, galactose, saccharose, sugar alcohol, lipid; or combinations thereof), and an adjuvant that is a polycation (e.g., polylysine or polyarginine) optionally modified with a sugar group. The adjuvant, according to one embodiment, can be a combination of a polycation and an immunostimulatory CpG or non-CpG oligodeoxynucleotide. One form of this adjuvant is the Intercell adjuvant IC31.

Yet another exemplary vaccine formulation that can be modified is described in U.S. Pat. No. 7,247,433 to Rose, which is hereby incorporated by reference in its entirety. For example, HPV virus-like particles could be administered with a pharmaceutically acceptable carrier and with or without E. coli LT R192G as the adjuvant.

The region of skin to be treated in accordance with the present invention is dependent on the intended purpose for delivery. For the vaccine delivery, it is intended that the vaccine be administered to a region of skin such as the upper arm, back, or the like.

As noted above, the present invention also relates to a transdermal drug formulation. The drug formulation includes a pharmaceutically suitable carrier; an effective amount of a therapeutic agent; and an H4R agonist.

The drug is present in a transdermal delivery device of the invention in a therapeutically effective amount, i.e., an amount effective to bring about a desired therapeutic result in the treatment of a condition. The amount that constitutes a therapeutically effective amount varies according to the particular drug incorporated in the device, the condition being treated, any drugs being coadministered with the selected drug, desired duration of treatment, the surface area of the skin over which the device is to be placed, and other components of the transdermal delivery device. Accordingly it is not practical to enumerate particular preferred amounts but such can be readily determined by those skilled in the art with due consideration of these factors. Generally, however, a drug is present in a transdermal device of the invention in an amount of about 0.01 to about 30 percent by weight based on the total weight of the drug storage material. In a preferred embodiment the drug is substantially fully dissolved, and the drug storage material is substantially free of solid undissolved drug.

The term “drug” and “therapeutic agent” are used interchangeably and are intended to have their broadest interpretation as any therapeutically active substance which is delivered to a living organism to produce a desired, usually beneficial, effect. In general, this includes therapeutic agents in all of the major therapeutic areas including, but not limited to, antiinfectives, antibiotics, antiviral agents, analgesics, fentanyl, sufentanil, buprenorphine, analgesic combinations, anesthetics, anorexics, antiarthritics, antiasthmatic agents, terbutaline, anticonvulsants, antidepressants, antidiabetic agents, antidiarrheals, antihistamines, antiinflammatory agents, antimigraine preparations, antimotion sickness, scopolamine, ondansetron, antinauseants, antineoplastics, antiparkinsonism drugs, cardiostimulants, dobutamine, antipruritics, antipsychotics, antipyretics, antispasmodics, gastrointestinal and urinary, anticholinergics, sympathomimetics, xanthine derivatives, cardiovascular preparations, calcium channel blockers, nifedipine, beta-blockers, beta-agonists, salbutamol, ritodrine, antiarrythmics, antihypertensives, atenolol, ACE inhibitors, diuretics, vasodilators, coronary, peripheral and cerebral, central nervous system stimulants, cough and cold preparations, decongestants, diagnostics, hormones, parathyroid hormone, growth hormone, insulin, hypnotics, immunosuppressives, muscle relaxants, parasympatholytics, parasympathomimetics, anti-oxidants, nicotine, prostaglandins, psychostimulants, sedatives, tranquilizers, skin acting anti-oxidants, caretenoids, ascorbic acid (vitamin C), vitamin E, anti wrinkling agents, retinoids, retinol (vitamin A alcohol), alpha-hydroxic acids, beta-hydroxy acid, salicylic acid, combination-hydroxy acids and poly-hydroxy acids, and hydrolyzed and soluble collagen, moisturizers, hyaluronic acid, anticellulite agents, aminophyllines, skin bleaching agents, retinoic acid, hydroquinone, peroxides, botanical preparations, extracts of aloe-vera, wild yam, hamamelitanin, ginseng, witch hazel, water, green tea, and combinations thereof.

The invention is also useful in the controlled delivery of polypeptide and protein drugs and other macromolecular drugs. These macromolecular substances typically have a molecular weight of at least about 300 daltons, and more typically a molecular weight in the range of about 300 to 40,000 daltons. In one embodiment, the therapeutic is at least 300 daltons in size. In another embodiment, the therapeutic is at least 500 daltons in size. In yet a further embodiment, the therapeutic is not less than 300 daltons in size.

Specific examples of peptides, and proteins and macromolecules in this size range include, without limitation, LHRH, LHRH analogs such as buserelin, gonadorelin, napharelin and leuprolide, GHRH, GHRF, insulin, insulotropin, heparin, calcitonin, octreotide, endorphin, TRH, NT-36 (chemical name: N=[[(s)-4-oxo-2-azetidinyl]carbonyl]-L-histidyl-L-prolinamid e), liprecin, pituitary hormones (e.g., HGH, HMG, HCG, desmopressin acetate, etc.), follicle luteoids, αANF, growth factors such as growth factor releasing factor (GFRF), βMSH, somatostatin, atrial natriuretic peptide, bradykinin, somatotropin, platelet-derived growth factor, asparaginase, bleomycin sulfate, chymopapain, cholecystokinin, chorionic gonadotropin, corticotropin (ACTH), epidermal growth factor, erythropoietin, epoprostenol (platelet aggregation inhibitor), follicle stimulating hormone, glucagon, hirulog, and other analogs of hirudin, hyaluronidase, interferon, insulin-like growth factors, interleukin-1, interleukin-2, menotropins (urofollitropin (FSH) and LH), oxytocin, streptokinase, tissue plasminogen activator, urokinase, vasopressin, desmopressin, ACTH analogs, ANP, ANP clearance inhibitors, angiotensin II antagonists, antidiuretic hormone agonists, antidiuretic hormone antagonists, bradykinin antagonists, CD4, ceredase, CSF's, enkephalins, FAB fragments, IgE peptide suppressors, IGF-1, neuropeptide Y, neurotrophic factors, oligodeoxynucleotides and their analogues such as antisense RNA, antisense DNA and anti-gene nucleic acids, opiate peptides, colony stimulating factors, parathyroid hormone and agonists, parathyroid hormone antagonists, prostaglandin antagonists, pentigetide, protein C, protein S, ramoplanin, renin inhibitors, thymosin alpha-1, thrombolytics, TNF, vaccines, vasopressin antagonist analogs, alpha-1 anti-trypsin (recombinant), and TGF-beta.

The present invention also encompasses artificial vesicles comprising the drug or vaccine formulations according to the present invention. Artificial vesicles include, for example, liposomes or micelles.

Another aspect of the present invention relates to a method of administering a transdermal drug formulation to a subject. The method involves applying a transdermal drug formulation of the present invention to an epithelial site on the subject.

Yet another aspect of the present invention relates to a method of administering a transdermal vaccine formulation to a subject. The method involves applying a transdermal vaccine formulation of the present invention to an epithelial site on the subject.

Accordingly, a further aspect of the present invention relates to a method of disrupting an epithelial barrier. The method involves administering to an epithelial site an amount of an H4R agonist that transiently disrupts tight junctions, thereby disrupting barrier formation at the epithelial site.

Yet another aspect of the present invention relates to a transdermal vaccine or drug delivery device or patch. The transdermal drug delivery device comprises a transdermal vaccine or drug formulation according to the present invention. In one embodiment, the transdermal vaccine or drug delivery patch includes a backing material, an adhesive material in contact with a first portion of the backing material; and a drug storage material comprising the transdermal vaccine or drug formulation, where the drug storage material is in contact with a second portion of the backing material. In one embodiment the patch also includes a releasable liner material to be removed upon application to the skin.

Any suitable backing material known in the art of transdermal patches (such as a breathable material) may be used in accordance with the present invention. The backing is flexible such that the device conforms to the skin. Exemplary backing materials include conventional flexible backing materials used for pressure sensitive tapes, such as polyethylene, particularly low density polyethylene, linear low density polyethylene, high density polyethylene, polyester, polyethylene terephthalate, randomly oriented nylon fibers, polypropylene, ethylene-vinyl acetate copolymer, polyurethane, rayon and the like. Backings that are layered, such as polyethylene-aluminum-polyethylene composites, are also suitable. The backing should be substantially inert to the ingredients of the drug storage material.

Adhesives suitable for use with the present invention will any dermatologically acceptable adhesive. Examples of dermatologically acceptable adhesives include, but are not limited to acrylics, natural and synthetic rubbers, ethylene vinyl acetate, poly(alpha-olefins), vinyl ethers, silicones, copolymers thereof and mixtures thereof. In an embodiment, the first adhesive layer includes a silicone adhesive (e.g., BIO-PSA 7-4302 Silicone Adhesive available commercially from Dow Corning®).

The transdermal patch may optionally include one or more release liners for storage or handling purposes. Many suitable release liners are known within the art. The release liner can be made of a polymeric material that may be optionally metallized. Examples of suitable polymeric materials include, but are not limited to, polyurethane, polyvinyl acetate, polyvinylidene chloride, polypropylene, polycarbonate, polystyrene, polyethylene, polyethylene terephthalate (PET), polybutylene terephthalate, paper, and combinations thereof. In certain embodiments, the release liner is siliconized. In other embodiments, the release liner is coated with fluoropolymer, such as PET coated with fluoropolymer (e.g., SCOTCHPAK™ 9744 from 3M™).

The drug storage material may be any dermatologically acceptable material suitable for use as a drug storage material or reservoir in a transdermal patch. For instance, the drug storage material may be a polymer. Examples of polymers include microporous polyolefin film (e.g., SOLUPOR® from SOLUTECH™), acrylonitrile films, polyethylnapthalene, polyethylene terephthalate (PET), polyimide, polyurethane, polyethylene, polypropylene, ethylene-vinyl acetate (EVA), copolymers thereof and mixtures thereof. In one embodiment, the polymer is EVA. In another embodiment, the polymer is EVA having a vinyl acetate content by weight in the range of about 4% to about 19%. In a preferred embodiment, the polymer is EVA having vinyl acetate content by weight of about 9%. The drug storage material may also include a heat-sealable material for attaching to other components. As an example, the heat-sealable permeable layer may be an EVA membrane, such as COTRAN™ 9702, available commercially from 3M™.

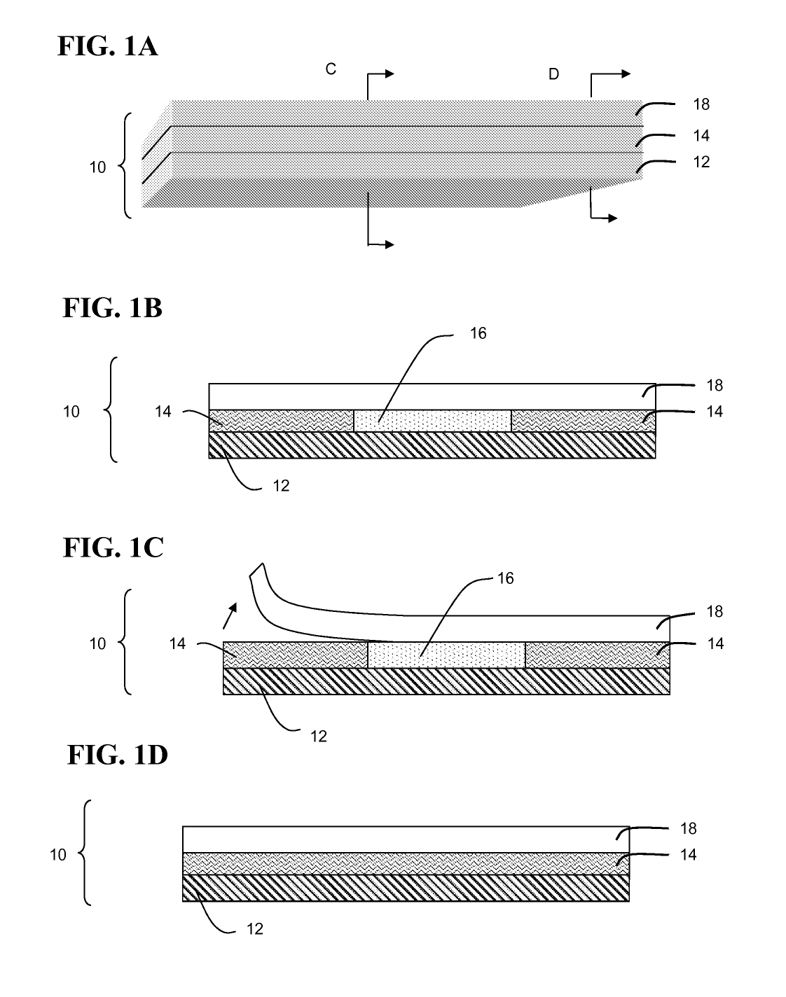

Referring now to FIGS. 1A to 1D, FIG. 1A is a perspective view of one embodiment of a transdermal patch according to the present invention. FIG. 1B is a cross-section of transdermal patch 10 along axis C of FIG. 1A. In one embodiment, transdermal patch 10 includes backing 12, adhesive material 14, and drug storage material 16. In addition, transdermal patch 10 may optionally include releasable liner 18, which is removed upon application to skin, as shown in FIG. 1C. FIG. 1D is a cross-sectional view of transdermal patch 10 along axis D of FIG. 1A.

Another aspect of the present invention relates to a method of enhancing epithelial barrier formation in a patient. The method involves administering to the patient at a site of epithelial disruption an amount of a formulation that comprises an H4R antagonist, thereby enhancing barrier formation at the site.

H4R antagonists that are suitable for use in accordance with the present invention include, but are not limited to, JNJ-7777120 (5-chloro-1H-indol-2-yl)(4-methyl-1-piperazinyl)-methanone), the benzofuro pyrimidine CZC-13788; the 2-amino pyrimidine PF-2988403; A-940894 (4-piperazin-1-yl-6,7-dihydro-5H-benzo[6,7]cyclohepta[1,2-d]pyrimidin-2-ylamine); A-987306 (cis-4-(Piperazin-1-yl)-5,6,7a,8,9,10,11,11a-octahydrobenzofuro[2,3-h]quinazolin-2-amine); (5-Chloro-7-methyl-1H-indol-2-yl)-(4-methyl-piperazin-1-yl)-methanone; (7-Amino-1H-indol-2-yl)-(4-methyl-piperazin-1-yl)-methanone; (5-Chloro-1H-indol-2-yl)-piperazin-1-yl-methanone; (5,7-Difluoro-1H-indol-2-yl)-(4-methyl-piperazin-1-yl)-methanone; (4-Methyl-piperazin-1-yl)-(3-methyl-4H-thieno[3,2-b]pyrrol-5-yl)-methanone; (2-Chloro-6H-thieno[2,3-b]pyrrol-5-yl)-(4-methyl-piperazin-1-yl)-methanone; (5-Chloro-1H-benzoimidazol-2-yl)-(4-methyl-piperazin-1-yl)-methanone; (5,6-Difluoro-1H-benzoimidazol-2-yl)-(4-methyl-piperazin-1-yl)-methanone; (2-Chloro-4H-thieno[3,2-b]pyrrol-5-yl)-(4-methyl-piperazin-1-yl)-methanone; (5-Chloro-1H-benzoimidazol-2-yl)-piperazin-1-yl-methanone; (2-Chloro-6H-thieno[2,3-b]pyrrol-5-yl)-(hexahydro-pyrrolo[1,2-a]pyrazin-2-yl)-methanone; 5-Methyl-1H-benzoimidazole-2-carboxylic acid (8-methyl-8-aza-bicyclo[3.2.1]oct-3-yl)-amide; (3-Bromo-4H-thieno[3,2-b]pyrrol-5-yl)-(3-methyl-piperazin-1-yl)-methanone; [5-Chloro-3-(4-chlorophenyl)-1-methyl-1H-indol-2-yl][4-(2-pyrimidinyl)-1-piperazinyl]methanone and combinations thereof. US Patent Application Publication No. 2004/0127395, Zhang et al., “The histamine H4 receptor: A novel modulator of inflammatory and immune disorders,” Pharmacol Ther. 113(3):594-606 (2006), and Lazewska et al., “Azines as Histamine H4 Receptor Antagonists,” Front Biosci. (Schol Ed) 4: 967-87 (2012), each of which is hereby incorporated by reference in its entirety. In certain embodiments, the H4R antagonist may be an H4R-specific antagonist.

The formulations for use according to this aspect of the present invention and those that follow may include one or more H4R antagonists. In one embodiment, the formulation includes a second H4R antagonist that is different from the first.

The formulation for use according to the present invention for use according to this aspect of the present invention and those that follow may also include an H1R antagonist.

Suitable H1R antagonists include, but are not limited to, azatidine, cetirizine, clozapine, amoxapine, loxapine, octoclothepin, ebastine, fexofenadine, hydroxyzine, loratidine, mizolastine, mepyramine, R-(+)-Terfenadine, astemizole, chlorpheniramine, cyproheptadine, desipramine, dexchlorpheniramine, diphenhydramine, doxepine, imipramine, ketotifen, mianserine, octoclothepin, ORG3770, promethazine, S-(−)-terfenadine, tripelennamine, triprolidine, and combinations thereof. Lim et al., “Evaluation of Histamine H1-, H2-, and H3-Receptor Ligands at the Human Histamine H4 Receptor: Identification of 4-Methylhistamine as the First Potent and Selective H₄ Receptor Agonist,” JPET 314(3): 1310-1321 (2005), which is hereby incorporated by reference in its entirety. In certain embodiments, H1R antagonists according to the present invention include H1R-specific antagonists.

The formulations according to this aspect of the present invention and those that follow may include a single histamine receptor antagonist that antagonizes more than one histamine receptor. For example, the formulations may include a single agent that is a H1R and H4R antagonist. In other embodiments, the formulations include multiple agents. For example, the formulation may include an agent that is an H1R antagonist and an agent that is an H4R antagonist.

In one embodiment, the formulation does not include an H2R antagonist.

The formulations according to the present invention may also include one or more additional agents that enhance epithelial barrier. For instance, the formulation may include an agent that increases claudin protein expression in epithelial cells (e.g., keratinocytes). For example, the formulation may include an agent that increases claudin-1, -4, and/or -23 expression in epithelial cells (e.g., keratinocytes). Agents that can increase claudin-1, -4, and/or -23 expression include, without limitation, interleukins, growth factors, synthetic or naturally occurring peptidoglycans (PGNs), toll-like receptor (TLR) ligands, pathogenic bacteria toxins or avirulence proteins, and peroxisome proliferator-activated receptor (“PPAR”) agonists.

Any suitable PPAR agonist may be used in accordance with the present invention. In one embodiment, the agonist is a PPARα or PPARγ agonist. PPARγ agonists are agents that bind to PPARγ and activate receptor-activated pathways. The PPARγ agonists can optionally have dual activity on other PPAR receptors (PPARα and PPARδ). Exemplary PPARγ agonists include, without limitation, cyclopentenone class prostaglandins, thiazolidinediones, glitazones, lysophosphatidic acid (“LPA”) or LPA derivatives (McIntyre et al., “Identification of an intracellular receptor for lysophosphatidic acid (LPA): LPA is a transcellular PPAR gamma agonist,” Proc. Natl. Acad. Sci. USA 100:131-136; (2003), which is hereby incorporated by reference in its entirety), tyrosine-based agonists, indole-derived agonists, and combinations thereof. A preferred member of the cyclopentenone class of prostaglandins is 15D-prostaglandin J₂. Preferred thiazolidinediones and/or glitazones include, without limitation, ciglitazone, troglitazone, pioglitazone, rosiglitazone, SB213068 (Smith Kline Beecham), GW1929, GW7845 (Glaxo-Wellcome), and L-796449 (Merck). Suitable tyrosine-based agonists include N-(2-benzylphenyl)-L-tyrosine compounds (Henke et al., “N-(2-benzylphenyl)-L-tyrosine PPARgamma Agonists: Discovery of a Novel Series of Patent Antihyperglycemic and Antihyperlipidemic Agents,” J. Med. Chem. 41:5020-5036 (1998), which is hereby incorporated by reference in its entirety. Suitable indole-derived agonists include those disclosed, e.g., in Hanks, et al., “Synthesis and Biological Activity of a Novel Series of Indole-derived PPARgamma Agonists,” Biorg. Med. Chem LLH. 9(23):3329-3334 (1999), which is hereby incorporated by reference in its entirety. Any other PPARγ agonists, whether now known or hereafter developed, can also be utilized in accordance with the present invention.

In addition to the use of PPARγ agonists per se, inducers of PPARγ agonists can also be utilized in accordance with the present invention. Inducers of PPARγ agonists are agents that induce an increase in the expression or production of a native PPARγ agonist. Exemplary inducers of PPARγ agonists include, without limitation, decorin or fragments thereof, enzymes that catalyze formation of prostaglandin D₂ precursor, and combinations thereof. Decorin is a small chondroitin/dermatan sulphate proteoglycan that binds the cytoline transforming growth factor beta (TGF-β) through its core protein. Preferred enzymes that catalyze formation of prostaglandin D₂ precursor are hematopoietic prostaglandin-D synthase and a lipocalin-form prostaglandin-D synthase. Any other inducers of PPARγ agonists, whether now known or hereafter developed, can also be utilized in accordance with the present invention.

PPAR-α agonists may also be used in accordance with the present invention and refers to compounds which activate PPARα. Examples include, but are by no means limited to, WY-14643, clofibrate, benzafibrate, fenofibrate, GW409544 and BM-17.0744.

In yet a further embodiment, claudin-1, -4, and/or -23 expression is increased by an agent other than a PPAR agonist.

Any of a number of suitable interleukins can be used to practice the present invention. Exemplary interleukins that can be used include, without limitation, IL-4, IL-13, IL-25, and IL-33. In one embodiment, IL-17A is the agent that increases claudin-4 expression.

Any of a number of suitable growth factors can be used to practice the present invention. Exemplary growth factors include, without limitation, epithelial growth factor (EGF), amphiregulin and transforming growth factor (TGF).

Any of a number of suitable TLR ligands can be used to practice the present invention. Exemplary TLR ligands that can be used include, without limitation, PAM3CSK4 (a synthetic triacylated lipopeptide, TLR2/TLR1 ligand), PAM2CSK4 (a synthetic diacylated lipoprotein-TLR2/TLR6 ligand), Poly I:C (a synthetic TLR3 ligand), MALP-2 and FSL-1 (Pam2CGDPKHPKSF). MALP-2, macrophage-activating lipopeptide-2, is induced via TLR2 and TLR6 signaling. FSL-1 is a synthetic lipoprotein derived from Mycoplasma salivarium similar to MALP-2, an M. fermentans derived lipopeptide (LP).

Any of a number of suitable PGNs can be used to practice the present invention. Exemplary PGNs include, without limitation, naturally occurring full-length peptidoglycan (PGN), muramyl dipeptide (MDP, a NOD2 ligand), O—(N-acetyl-β-D-glucosaminyl)-(1→4)-N-acetylmuramyl-L-alanyl-D-isoglutamine, O—(N-acetyl-β-muramyl-L-alanyl-D-isoglutamine)-(1→4)-N-acetyl-D-glucosamine, meso-diaminopimelic acid (meso-DAP), glucosaminyl-N-acetyl)-β-(1→4)-(anhydro)muramyl-N-acetyl-L-alanyl-γ-D-glutaminyl-meso-DAP-D-alanine, glucosaminyl-N-acetyl)-β-(1→4)-muramyl-N-acetyl-L-alanyl-γ-D-glutaminyl-meso-DAP-D-alanine, MurNAc-L-Ala-D-isoGln-L-Lys-D-Ala-D-Ala (MPP), MurNAc-L-Ala-D-isoGln-L-Lys-D-Ala, MurNAc-L-Ala-D-isoGln-(2S,6R)-Dap-D-Ala-D-Ala (MPP-Dap), GlcNAc-MurNAc(1,6-anhydro)-L-Ala-D-isoGlu-(2S,6R)-Dap-D-Ala (TCT), GlcNAc-MurNAc-L-Ala-D-isoGln-L-Lys-D-Ala)₂ (T-4P₂), and various PGN derivatives described in Wolfert et al., “Modification of the Structure of Peptidoglycan Is a Strategy To Avoid Detection by Nucleotide-Binding Oligomerization Domain Protein 1,” Infection and Immunity, 75(2):706-713 (2007), PCT Application Publ. No. WO 2006/113792, US Application Publ. No. 20090214598, and US Application Publ. No. 20070041986, each of which is hereby incorporated by reference in its entirety.

Any of a number of suitable pathogenic bacteria toxins or avirulence proteins can be used in practicing the present invention. Exemplary toxins include, without limitation, Vibrio cholera zonula occludens toxin (Zot) and active fragments thereof (e.g., AT1002) (Song et al., “Effect of the Six-Mer Peptide (AT1002) Fragment of Zonula Occludens Toxin on the Intestinal Absorption of Cyclosporine A,” Int. J. Pharm. 351:8-14 (2008), which is hereby incorporated by reference in its entirety). Exemplary avirulence proteins include, without limitation, Salmonella AvrA (see PCT Application Publ. No. WO 2009/149191, which is hereby incorporated by reference in its entirety).

Pharmaceutical compositions including formulations according to this aspect of the invention and those that follow may include a suitable carrier, as described above. The pharmaceutical compositions may be formulated for administrating topically (as described above with respect to transdermal or transmucosal formulations) or by any other means suitable. For example, the pharmaceutical compositions may be formulated for administration orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes. The formulations may conveniently be presented in unit dosage form and may be prepared by any of the methods well known in the art of pharmacy.

A patient in need of enhanced epithelial barrier formation includes one having a disease or disorder mediated by histamine. Histamine generally comes from mast cells, basophils, and eosinophils. Typically the release of histamine is in response to crosslinking of IgE which is bound to high affinity IgE receptors (FceR1), but release can also occur in non-immunologic ways, e.g., certain amines and alkaloids, including such drugs as morphine, and curare alkaloids, can displace histamine in granules and cause its release. Antibiotics like polymyxin are also found to stimulate histamine release. Bacteria also are capable of producing histamine using histidine decarboxylase enzymes unrelated to those found in animals. A non-infectious form of foodborne disease, scombroid poisoning, is due to histamine production by bacteria in spoiled food, particularly fish. Fermented foods and beverages naturally contain small quantities of histamine due to a similar conversion performed by fermenting bacteria or yeasts. For example, sake contains histamine in the 20-40 mg/L range; wines contain it in the 2-10 mg/L range. Jayarajah et al., “Analysis of Neuroactive Amines in Fermented Beverages Using a Portable Microchip Capillary Electrophoresis System,” Anal. Chem. 79(21):8162-8169 (2007), which is hereby incorporated by reference in its entirety.

Histamine-mediated diseases treatable in accordance with the present invention include those in which the signs and symptoms are primarily or largely due to histamine. Such diseases include, but are not limited to all allergic diseases (Asthma, allergic rhinitis (i.e., hay fever), allergic rhinoconjunctivitis, allergic sinusitis, atopic dermatitis, food allergy, eosinophilic esophagitis, and acute and chronic urticaria); all forms of mastocytosis; anaphylaxis; ingestion of opiates (morphine, codeine, etc), antibiotics (polymyxin), or spoiled fish (e.g. mackerel, tuna, bluefish, mahi-mahi, bonito, sardines, anchovies) which can lead to Scombroid poisoning; tumors that produce histamine (Graff et al., “Expression of Histidine Decarboxylase and Synthesis of Histamine by Human Small Cell Lung Carcinoma,” Am. J. Path. 160(5):1561-1565 (2002), which is hereby incorporated by reference in its entirety); hypereosinophilic diseases (for example, those described in Valent et al., “ICON: Eosinophil Disorders,” World Allergy Organ J. 5(12):174-81 (2012), which is hereby incorporated by reference in its entirety); inflammatory bowel disease (histamine release from mast cells); many drug reactions; wound repair; and hereditary Angioedema.

Accordingly, the patient to be treated in accordance with the invention includes one with a skin inflammatory condition. Skin inflammatory conditions include, but are not limited to, Atopic dermatitis, allergic eczema, psoriasis, contact dermatitis, xerosis, and combinations thereof. Skin inflammatory conditions also include dry skin. The patient may also have an epithelial condition mediated by an inflammatory condition (e.g., asthma, hay fever, rhinitis, and combinations thereof). Such an inflammatory condition also includes inflammatory intestinal conditions including, for example, Inflammatory Bowel Disease, Celiac Disease, or gastroenteritis.

Suitable regions of the epithelia to be treated in accordance with this aspect of the invention and those that follow include those that have reduced epithelial function. Regions having reduced epithelial function may include any region of injury, which communicates with the atmosphere, by direct exposure. In one embodiment, the region of epithelia is the skin. The region may be a skin site that may be intact (e.g., normal skin) or may be compromised, defined as skin that is damaged or that lacks at least some of the stratum corneum (e.g., skin damaged by exposure to the agent in question, another agent, the presence of a pathological condition such as a rash or contact dermatitis, a physical trauma such as a cut, wound, or abrasion, a underdeveloped skin such as occurs in a preterm infant, conditions in which either all or part of the epidermis is exposed, conditions in which part of the dermis has been removed such as partial thickness wounds encountered in resurfacing procedures such as chemical peels, dermabrasions, and laser resurfacing, etc.). The region of epithelia may also include other areas of epithelia, e.g., mucosa (e.g., nasal, buccal, vaginal, oral, intestinal, rectal, airway, etc.).

Open wounds or denudated areas are also included. Open wounds include, but are not limited to, decubital ulcers, dehiscence wounds, acral lick dermatitis (acral lick granulomas in animals), lacerations, and both traumatic and surgical wounds. By ulcer or cutaneous ulcer it is meant a break in the continuity of the epidermis with a loss of substance and exposure of underlying tissue. The region may also include a burn wound, which may include a surface wound ranging from first to third degree burn and ranging from affecting 0.1% to 99.9% body surface area. Exemplary regions include, but are not limited to, those regions disrupted by burn (e.g., thermal or chemical), cutaneous ulcer, severe Stevens-Johnson Syndrome, toxic epidermal necrolysis, autoimmune blistering disorders, or those that having a region of denudation.

Any tissue graft or tissue scaffold or heterologous or autologous epidermal or epithelial sheets suitable for re-epithelialization may be used in accordance with the present invention. Exemplary tissue grafts include epithelial grafts that include any natural epithelial (e.g., skin) substitutes such as xenografts, allografts, and autografts. Exemplary tissue scaffolds include, but are not limited to, epidermal or epithelial sheets, collagen-based matrices, natural polymers (e.g., chitosan, fibrin, elastin, gelatin, and hyaluronic acid), synthetic polymer scaffolds, and electrospun biomimetic nanofibrous scaffolds. Zhong et al., “Tissue Scaffolds for Skin Wound Healing and Dermal Reconstruction,” WIREs Nanomedicine and Nanotechnology 2:510-525 (2010), which is hereby incorporated by reference in its entirety. The tissue scaffold may be in any suitable form including, but not limited to, that of a gel, sheet, lattice or sponge. The scaffold may also be formed so as to include the formulation and/or agents according to the present invention. This will allow the agent to be released at the site of scaffold use where it can affect tight junction formation between epithelial cells (e.g., keratinocytes). The scaffold may also include or be administered with epithelial cells. For example, the scaffold may include or be administered with skin cells (e.g., keratinocytes, fibroblasts, or both).

Pharmaceutical formulations also include mucoadhesive formulations. Accordingly, the use various polymers, particularly mucoadhesive polymers, in pharmaceutical compositions according to the present invention is contemplated. Typically, mucoadhesive polymers for use in accordance with the present invention are natural or synthetic macromolecules which adhere to wet mucosal tissue surfaces by complex, but non-specific, mechanisms. Mucoadhesive polymers include, for example, acrylic acid polymers, methyl vinyl/maleic acid polymers and polyvinyl polymers. Exemplary mucoadhesive formulations are described in US 2008/0021103 to Abu-izza et al., WO/2013/188979 to Gu et al., WO/2008/153966 to Hirt et al., and WO/2007/047948 to Quay et al., which are each incorporated by reference in their entirety.

In addition to these mucoadhesive polymers, pharmaceutical compositions according to the present invention also may include bioadhesives that adhere directly to a cell surface, rather than to mucus, by means of specific, including receptor-mediated, interactions. One example of bioadhesives that function in this specific manner is the group of compounds known as lectins. These are glycoproteins with an ability to specifically recognize and bind to sugar molecules, e.g., glycoproteins or glycolipids, which form part of intranasal epithelial cell membranes and can be considered as “lectin receptors.” In certain embodiments, bioadhesive materials for enhancing intranasal delivery of biologically active agents comprise a matrix of a hydrophilic, e.g., water soluble or swellable, polymer or a mixture of polymers that can adhere to a wet mucous surface. These adhesives may be formulated as ointments, hydrogels, thin films, and other application forms. Often, these adhesives have the biologically active agent mixed therewith to effect slow release or local delivery of the active agent. Some are formulated with additional ingredients to facilitate penetration of the active agent through the mucosa, e.g., into the circulatory system of the individual.

Accordingly, mucoadhesive compositions may also include other compounds that facilitate transmucosal delivery (e.g., a degradative enzyme inhibitor). Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)—Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil—elastatinal (anti-elastase); Chitosan—antipain (anti-trypsin); Poly(acrylic acid)—bacitracin (anti-aminopeptidase N); Chitosan—EDTA (anti-aminopeptidase N, anti-carboxypeptidase A); Chitosan—EDTA—antipain (anti-trypsin, anti-chymotrypsin, anti-elastase). Any inhibitor that inhibits the activity of an enzyme to protect the biologically active agent(s) may be usefully employed in the compositions and methods of the invention. Useful enzyme inhibitors for the protection of biologically active proteins and peptides include, for example, soybean trypsin inhibitor, exendin trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovoniucoid-enzyme, gabaxate mesylate, alpha1-antitrypsin, aprotinin, amastatin, bestatin, puromycin, bacitracin, leupepsin, alpha2-macroglobulin, pepstatin and egg white or soybean trypsin inhibitor. These and other inhibitors can be used alone or in combination.

Yet another aspect of the present invention relates to a method of promoting epithelial function in an individual having compromised or immature epithelial function. The method involves providing a formulation comprising H4R antagonist, an H1R antagonist, or a combination thereof that enhances tight junction formation between epithelial cells; and administering the formulation to a region of epithelia on an individual having reduced epithelial function at the region, thereby enhancing tight junction formation between epithelial cells and promoting epithelial function in the individual.

In one embodiment, the region of epithelia is the skin and the epithelial cells are keratinocytes. The individual may be an infant or an individual of any age that has a defect in skin barrier or a genetic or acquired condition for which TJ defects are a component of the disease. Examples of such conditions are noted above. In one embodiment, the individual is the individual is an infant, has a cutaneous ulcer, has a region of denudation, has superficial loss of epidermis, or an increase in trans epidermal water loss.

The individual having compromised or immature epithelial function includes a full-term infant, a preterm infant, a low-birth-weight infant, or a very-low-birth-weight infant. As used herein, the terms “preterm” or “preterm infant” may include low-birth-weight infants or very-low-birth weight infants. Low-birth-weight infants are those born from about 32 to about 37 weeks of gestation or weighing from about 3.25 to about 5.5 pounds at birth. Very-low-birth-weight infants are those born before about 32 weeks of gestation or weighing less than about 3.25 pounds at birth. Thus, preterm infants may include infants born before about 37 weeks gestation and/or those weighing less than about 5.5 pounds at birth.

According to one embodiment, the formulation is applied to the region of the epithelia (e.g., skin or mucosa) up to several times daily. According to another embodiment, the formulation is applied to the region of epithelia once daily. According to further embodiments, the formulation is applied to the region of epithelia periodically (e.g., every other or every third day).

Suitable H4R and H1R antagonists, as well as formulations and pharmaceutical compositions are described above. In one embodiment, the formulation may include one or more H4R and H1R antagonists. In one embodiment, the formulation does not include an H2R antagonist.

The formulations according to this aspect of the present invention and those that follow may also include one or more additional agents that enhance epithelial barrier. For instance, the formulation may include an agent that increases claudin protein expression in epithelial cells (e.g., keratinocytes). The claudin may be, for example, claudin-1, -4, and/or -23. In one embodiment the agent is selected from a PPARγ agonist, a PPARα agonist, or a combination thereof. In yet a further embodiment, the agent is one that is other than a PPAR agonist, including interleukins, growth factors, synthetic or naturally occurring peptidoglycans (PGNs), toll-like receptor (TLR) ligands, and pathogenic bacteria toxins or avirulence proteins, as described above. In one embodiment, the formulation includes a second H1R antagonist, a second H4R antagonist, an agent that increases claudin-1, -4, and/or -23 expression in epithelial cells, or a combination thereof.

Yet a further aspect of the present invention relates to a method of inhibiting pathogen infection or local spread of infection in the epithelia. The method comprises providing a formulation comprising an H4R antagonist, an H1R antagonist, or a combination thereof; and applying to a region of epithelia on an individual that is susceptible to pathogen infection an amount of the formulation that is effective to enhance epithelial barrier formation at the application site, thereby rendering the application site less susceptible to pathogen infection or local spread of infection. Suitable areas of epithelia treatable in accordance with this aspect of the present invention are described above.

In one embodiment, the area of epithelia is the skin. Accordingly, one embodiment of the present invention relates to a method of inhibiting pathogen infection or local spread of infection in the skin. The method comprises providing a formulation comprising an H4R antagonist, an H1R antagonist, or a combination thereof; and applying to a region of skin on an individual that is susceptible to pathogen infection an amount of the formulation that is effective to enhance epidermal barrier formation at the application site, thereby rendering the application site less susceptible to pathogen infection or local spread of infection.

In one embodiment, the area of epithelia is the mucosa (e.g., nasal, buccal, vaginal, oral, intestinal, rectal, airway, etc.). Accordingly, one embodiment of the present invention relates to a method of inhibiting pathogen infection or local spread of infection in the mucosa. The method comprises providing a formulation comprising an H4R antagonist, an H1R antagonist, or a combination thereof; and applying to a region of mucosa on an individual that is susceptible to pathogen infection an amount of the formulation that is effective to enhance epithelial barrier formation at the application site, thereby rendering the application site less susceptible to pathogen infection or local spread of infection.

Suitable H4R and H1R antagonists, as well as formulations and pharmaceutical compositions are described above. In one embodiment, the formulation may include one or more H4R and H1R antagonists. In one embodiment, the formulation includes a second H1R antagonist, a second H4R antagonist, an agent that increases claudin-1, -4, and/or -23 expression in epithelial cells (e.g., keratinocytes), or a combination thereof. In one embodiment, the formulation does not include an H2R antagonist.

In carrying out the method of inhibiting pathogen infection or local spread of infection in the epithelia (e.g., skin or mucosa), the region of epithelia to be treated is generally any region of epithelia that is susceptible to pathogen infection. By way of example, the region of epithelia (e.g., skin or mucosa) may include at least a portion of the individual's hand, foot, face, or genitalia. Other regions of exposed epithelia (e.g., skin or mucosa) can also be treated in accordance with the present invention. Application of the compositions can be carried out as described above, preferably as part of a daily routine (i.e., after bathing) to inhibit virus infection or local spread thereof. In the case of HSV reactivation it may be used prior to increased sun exposure when the HSV infection typically reactivates in sun-exposed regions of the body.

The pathogen targeted according to the present invention may be a virus. Any virus that is transmitted or infects via the epidermis can be targeted by the methods of the present invention. Exemplary viruses whose infection can be inhibited or blocked include, without limitation, HIV-1, vaccinia virus, varicella zoster virus, herpes simplex viruses (HSV), papillomavirus (e.g., HPV), molluscum contagiosum or Variola (Smallpox) or monkeypox.

The pathogen targeted according to the present invention may be a bacterial pathogen. Any bacterium that is transmitted or infects via the epidermis can be targeted by the methods of the present invention. Exemplary bacterial pathogens whose infection can be inhibited or blocked include, but are not limited to, infections caused by gram-positive and gram-negative bacteria including Staphylococcus, Staphylococcus aureus (including MRSA and MSSA), Hemophilus, Hemophilus influenzae, Pseudomonas, Pseudomonas aeruginosa, Streptococcus, Streptococcus pneumoniae, Streptococcus Group A, Group B, Group C, Group D, Group G, Mycobacterium, Mycobacterium tuberculosis, Atypical Mycobacterium, Clostridium, and Enterobacteriaceae.

According to one embodiment, individuals to be treated for inhibiting pathogen infection or local spread of infection can be healthy individuals having normal TJ protein function.

According to another embodiment, individuals to be treated for inhibiting pathogen infection or local spread of infection can be individuals that have compromised TJ protein function, particularly with respect to claudin-1, -4, and/or -23 expression levels or activity. Individuals that have compromised TJ protein function can include, without limitation, those that are identified as having atopic dermatitis (AD, or eczema), psoriasis, contact dermatitis, drug eruptions, Darier's Disease, Netherton's Syndrome, Hyper IgE syndrome, Wiskott Aldrich syndrome, neonatal sclerosing cholangitis associated with ichthyosis, or two or more of the above. By virtue of their compromised TJ function, these individuals are particularly susceptible to virus infection.

### EXAMPLES

The following examples are provided to illustrate embodiments of the present invention but are by no means intended to limit its scope.

### Materials and Methods for Example 1

### Culture of Primary Human Keratinocytes

Human keratinocytes were isolated from neonatal foreskin as previously described (Poumay et al., “Basal Detachment of the Epidermis Using Dispase: Tissue Spatial Organization and Fate of Integrin alpha 6 beta 4 and Hemidesmosomes,” J Invest Dermatol 102:111-7 (1994), which is hereby incorporated by reference in its entirety). PHK were cultured in Keratinocyte-SFM (Invitrogen/Gibco, Grand Island, N.Y.) with 5 ng/ml human recombinant Epidermal Growth Factor 1-53 (EGF 1-53), 50 μg/ml Bovine Pituitary Extract (BEP), 1% Pen/Strep, and 0.2% Amphotericin B (Invitrogen/Gibco). To induce terminal differentiation PHK were grown in DMEM (Invitrogen/Gibco) with 1% Pen/Strep and 0.2% Amphotericin B (Invitrogen/Gibco). Where it is indicated, 10% heat-inactivated bovine serum (Invitrogen) was added to the differentiation media. Histamine (1-100 μM) and antagonists were added, alone or in combination, to the culture media from the time of differentiation and replaced every 48 hr with media change. Selective antagonists were used for each receptor: H1R (Cetirizine 10 μM), or H4R (JNJ7777120, 10 μM).

### TJ Assessments: Transepithelial Electric Resistance (TEER) and Paracellular Flux

TJ integrity in cultured primary keratinocytes was evaluated using two complementary assays, TEER and paracelluar flux, as previously described (De Benedetto et al., “Tight Junction Defects in Patients with Atopic Dermatitis,” J Allergy Clin Immunol 127:773-86 e1-7 (2011), which is hereby incorporated by reference in its entirety). Briefly, TEER was measured using an EVOMX voltohmmeter (World Precision Instruments, Sarasota, Fla.). To evaluate the paracellular flux of PHK, 0.02% Fluorescein (FITC) Sodium (Fluka, St. Louis, Mo.) in PBS (Invitrogen/Gibco) was added to the upper chamber. Samples were collected from the lower chamber after 30 minutes incubation for PHK culture or 10 and 20 minutes for skin explant. For each experiment paracellular flux was normalized to an empty filter.

### Micro-Snapwell™ Barrier Function Assay on Human Skin Explant.

Skin samples were mounted on filter supports (Whatman Nuclepore Track-Etch Membrane; GE Healthcare Biosciences, Pittsburgh, Pa.) with the epidermal side oriented upward, and sandwiched between two sterile custom made Plexiglas discs with an opening of 3 mm and placed in modified Snapwell™ chambers (Corning, Corning, N.Y., USA). Samples were submerged in DMEM media and kept at 37° C., 5% CO₂ for 30 min. Fresh media with histamine (10 or 100 μM) or media alone were then added to both sides of the transwell. TEER and Paracellular flux of fluorescein of skin explant were measured at 24 hr.

### Immunoblotting

PHK were lysed on ice in RIPA lysis buffer [50 mM Tris-HCl (pH 7.4), 150 mM NaCl, 5 mM EDTA, 1% NP-40, 0.5% sodium deoxycholate, and 2% SDS] with 1:100 Protease Inhibitor Cocktail (Sigma), 1:100 Phosphatase Inhibitor Cocktail (SigmaCells) for 15 min. After transferring cell lysates into 1.5-ml tube, the samples were further incubated for 30 min at 4° C. The samples were heated at 95° C. for 10 min and then centrifuged at 14,000 rpm for 15 min. The protein concentration of all samples was determined by BCA (Pierce). Forty μg of protein in NuPage LDS Sample Buffer (Invitrogen) was applied to 4-12% NuPage Bis-Tris gels (Invitrogen). Electrophoresis was performed under reducing conditions with MES SDS Buffer (Invitrogen) followed by Western blotting. Membranes were incubated in blotting solution (5% non-fat dry milk in PBS+0.05% Tween-20) at RT for 1 hr and then incubated with a monoclonal Filaggrin antibody ([SPM181], ab17808, abcam, Cambridge, Mass.) at 1:500 dilution. A HRP-linked secondary antibody (GE Healthcare, Fairfield, Conn.) was used to detect pro-Filaggrin and subsequently monoclonal beta-actin antibody conjugated with Horseradish peroxidase (sc-47778 HRP, Santa Cruz Biotechnology, Santa Cruz, Calif.) at 1:10000 dilution was used to detect beta-actin with ECL (GE Healthcare) to visualize bands by autoradiography with Kodak BioMax MR film.

Quantitative PCR (qPCR)

AD and non-atopic control subjects underwent a 5-mm punch biopsy of their non-sunexposed forearm (nonlesional). AD subjects also underwent a 5-mm punch biopsy at an adjacent lesional site. Skin samples were immersed in TRI-reagent (Sigma-Aldrich, St. Luis, Mo.) and stored at −80° C. For qPCR analysis total RNA was extracted from skin samples using the QIAshredder spin column (Qiagen, Valencia, Calif.) and RNeasy RNA isolation kits (Qiagen).

QPCR was performed using the iScript™ cDNA Synthesis kit and iQ™ SYBER Green Supermix assay system (Bio-Rad Laboratories, Hercules, Calif.). All PCR amplifications were carried out in triplicate on an iQ5 Multicolor real-time PCR detection system (Bio-Rad). Primers were designed and synthesized by Integrated DNA Technologies. Relative gene expression was calculated by using the 2^{-ΔΔCt} method. The normalized Ct value of each sample was calculated using GAPDH as an endogenous control gene

### Example 1

### Histamine Receptors H4R and H1R Control TJ Barrier Function

A growing body of evidence suggests that Atopic Dermatitis (AD) develops as the consequence of an acquired or genetic defect in skin barrier. Recent studies have highlighted that histamine inhibits human keratinocyte terminal differentiation and promotes proliferation. Gschwandtner et al., “Histamine Suppresses Epidermal Keratinocyte Differentiation and Impairs Skin Barrier Function in a Human Skin Model,” Allergy 68:37-47 (2013); Glatzer et al., “Histamine Induces Proliferation in Keratinocytes from Patients with Atopic Dermatitis Through the Histamine 4 Receptor,” J. Allergy Clin Immunol 132(6):1358-67 (2013), which are hereby incorporated by reference in their entirety. Histamine has also been shown to disrupt Tight Junction (TJ) in endothelial cells, but little is known about its actions on epidermal TJs. In this example, the effect of histamine and selected histamine receptor (H1R, H2R and H4R) antagonists on epidermal TJ function and composition was investigated. The expression of HRs in the skin of AD and non-atopic controls was also quantified.

Ca⁺²-differentiated primary human keratinocytes (PHK) and epidermal explants were treated with histamine (1-100 μM) to determine the effect this had on TJ function. TJ integrity was assessed by trans-epithelial electrical resistance (TEER) and paracellular fluorescein flux (permeability). Selective antagonists were used for each receptor: H1R (Cetirizine 10 μM), H2R (Cimetidine, 100 μM), or H4R (JNJ7777120, 10 μM). Keratinocyte differentiation was assessed by examining filaggrin, loricrin and keratin 10 expression by Western blot. Expression of HRs were evaluated by qPCR in skin biopsies taken from lesional and nonlesional sites in AD subjects (n=6-8) and non-atopics (n=10).

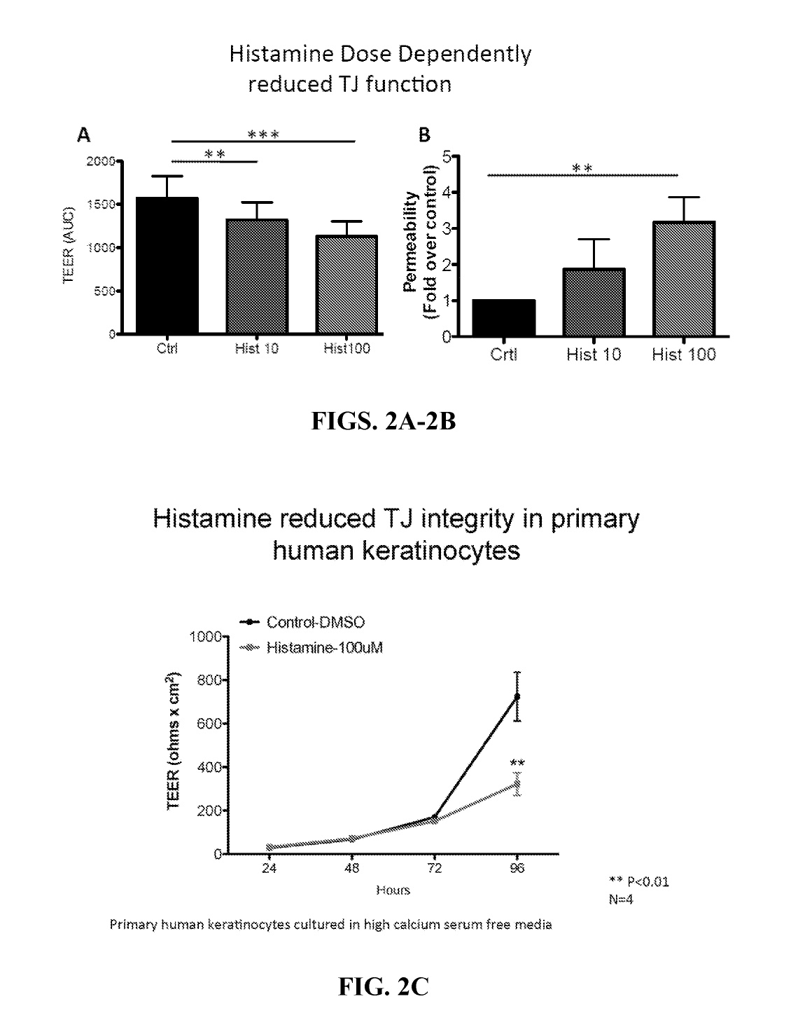

Results are shown in FIGS. 2-5. FIGS. 2A-2C are graphs showing results that demonstrate, in cultured primary human keratinocytes (PHK), histamine reduced TJ integrity. Histamine dose-dependently reduced (FIG. 2A) transepithelial electric resistance (TEER) and it (FIG. 2B) enhanced Permeability to Fluorescein (96 hrs). TEER is shown as Mean Area under the curve of 0-120 h time points (AUC) ±SEM on n=10 experiments; Permeability is shown as Mean Fold of control (FOC) ±SEM of n=9. *p<0.05; **p<0.001, ***p<0.0001. FIG. 2C also shows results demonstrating that in PHK, histamine significantly reduced trans epithelial electric resistance (TEER) peak observed at 96 hours post-stimulation. PHK were differentiated in DMEM serum free media ±histamine (100 μM). TEER is shown as Mean±SEM of n=4 experiments. **p<0.01

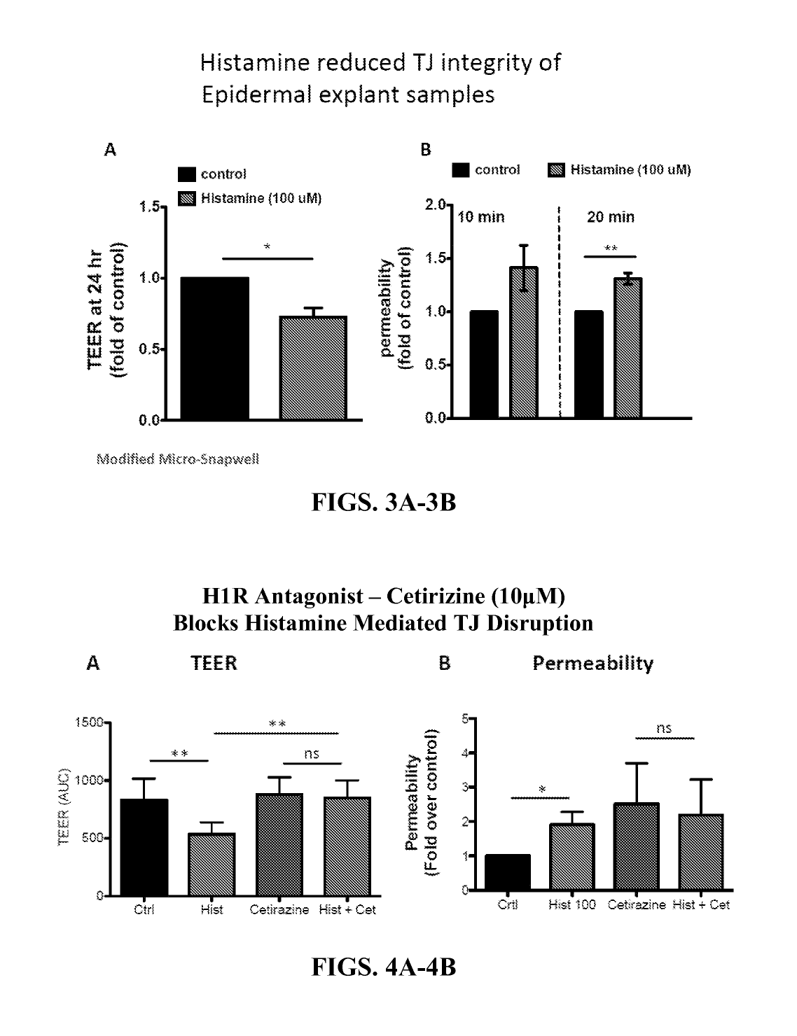

FIGS. 3A and 3B are bar graphs showing results that demonstrate, in an ex-vivo model, using skin explanted and the adapted Microsnap-well system, histamine (100 μM) (FIG. 3A) reduced TEER (0.7 fold) and (FIG. 3B) enhanced Fluorescein permeability flux (20 minutes time point; 1.3 fold) 24 hours post stimulation. TEER is shown as Mean Fold of control (FOC) ±SEM on n=3; permeability is shown as Fold of control (FOC) Mean±SEM of n=3. *p<0.05; **p<0.001, ***p<0.0001.

FIGS. 4A and 4B are bar graphs showing results that demonstrate, in cultured primary human keratinocytes, Cetirizine (Cet, 10 μM—H1R antagonist) blocks histamine-mediate (FIG. 4A) TEER reduction (n=4) and (FIG. 4B) permeability enhancement (n=9; 96 hours). TEER is shown as Mean Area under the curve 0-96 hours (AUC) ±SEM; permeability is shown as Mean Fold of control (FOC) ±SEM. *p<0.05; **p<0.001, ***p<0.0001.

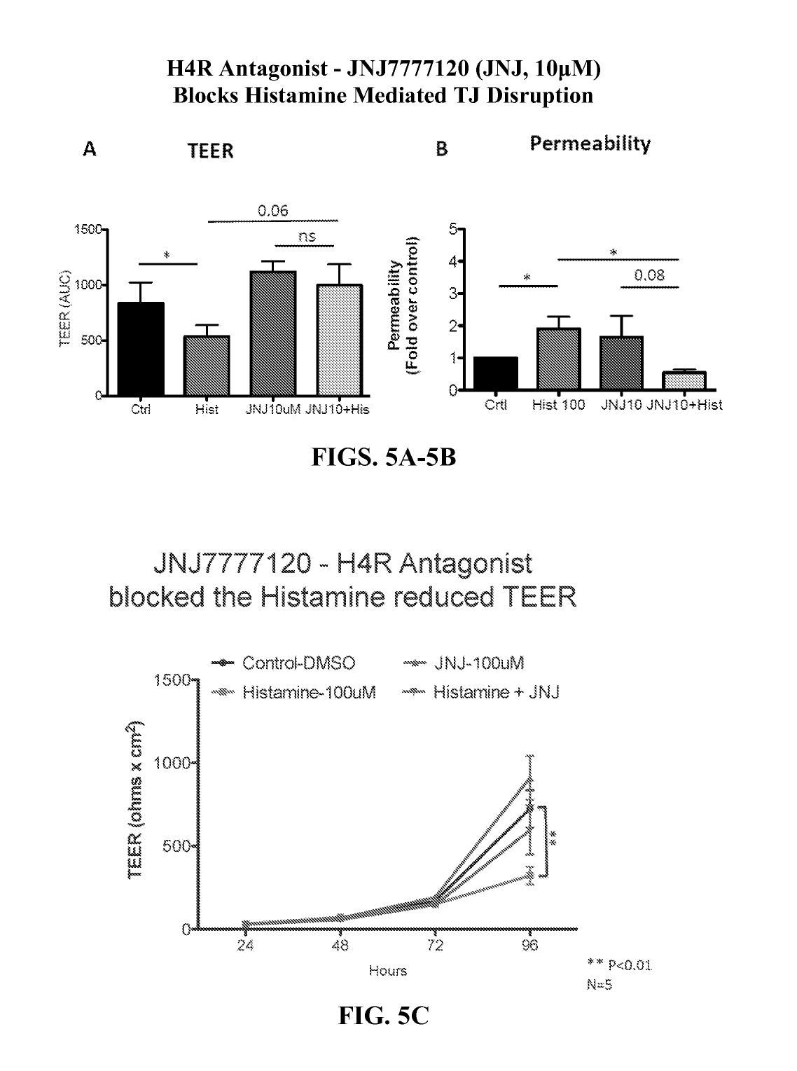

FIGS. 5A-5C are graphs showing results that demonstrate, in cultured primary human keratinocytes, JNJ7777120 (JNJ, 10 μM—H4R antagonist) blocks histamine-mediate (FIG. 5A) TEER reduction (n=4) and (FIG. 5B) permeability enhancement (n=9; 96 hours). As also demonstrated by the results shown in FIG. 5C, in cultured primary human keratinocytes, JNJ7777120 (JNJ, 10 μM—H4R antagonist) blocks histamine-mediate TEER reduction over the course of 120 hours. TEER is shown as Mean Area under the curve (AUC) ±SEM; permeability is shown as Mean Fold of control (FOC) ±SEM. *p<0.05; **p<0.001, ***p<0.0001.

As noted above, histamine significantly reduced TJ barrier function. In cultured PHK, a dose dependent reduction of TEER (10 and 100 μM, P<0.001, n=10) and enhancement of permeability (100 μM, P<0.001, n=9) was observed (FIGS. 2A-2B). FIG. 2C shows additional results demonstrating that in PHK, histamine significantly reduced TEER peak. Also, in PHK, H1R and H4R, but not H2R, antagonists blocked histamine-mediated TEER reduction (FIGS. 4A-4B and 5A-5C).

Using the ex-vivo model, it was confirmed that histamine (100 μM) reduced TEER (0.7 fold, P<0.05, n=3) and enhanced fluorescein permeability flux (1.3 fold, P<0.05, n=3) in epidermal explants (FIGS. 3A and 3B).

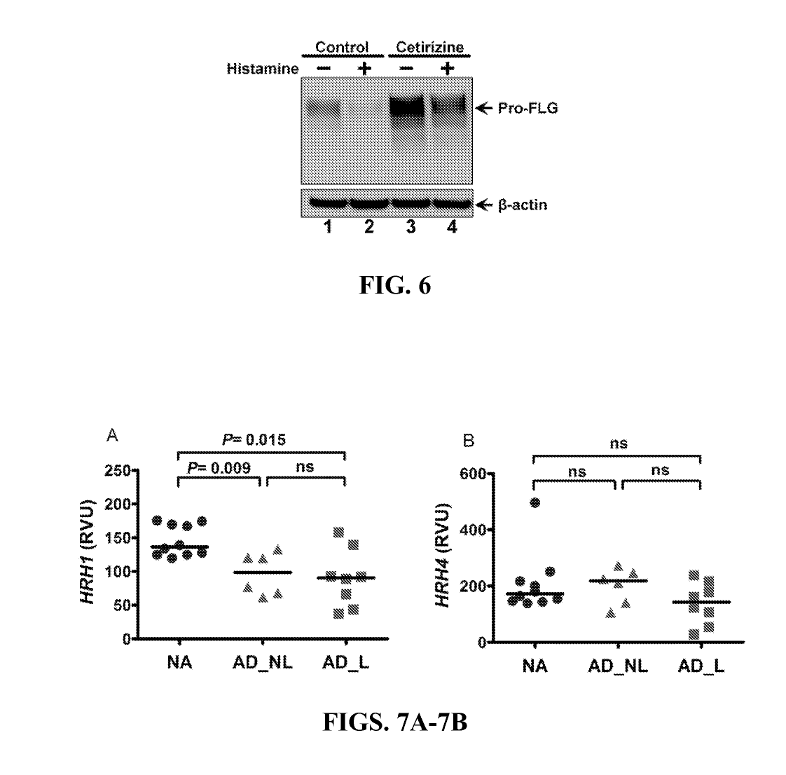

In addition, it was confirmed that histamine selectively reduced filaggrin. Only H1R antagonist was able to prevent the histamine-mediated reduction of filaggrin expression (FIG. 6). Interestingly, H1R was reduced in AD skin lesional and non-lesional (P<0.01) (FIG. 7A). No significant changes in H4R expression were found (FIG. 7B).

These studies revealed that histamine contributes to epidermal barrier impairment observed in AD skin, by reducing TJ integrity (H1R and H4R dependent) and filaggrin expression (H1R dependent).

Although the invention has been described in detail for the purpose of illustration, it is understood that such detail is solely for that purpose, and variations can be made therein by those skilled in the art without departing from the spirit and scope of the invention which is defined in the following claims. Further, even though specific combinations may not be explicitly recited herein, it is to be understood by persons of skill in the art that such combinations of features are intended to be encompassed by the present disclosure for the recited products and methods.

1. A transdermal drug formulation comprising:
a pharmaceutically suitable carrier;
an effective amount of a therapeutic agent; and
a histamine type 4 receptor (“H4R”) agonist.

2. The transdermal drug formulation according to claim 1, wherein the H4R agonist is selected from the group consisting of endogenous H4R ligands, histamine analogs, and combinations thereof.

3. The transdermal drug formulation according to claim 1, wherein the H4R agonist is selected from the group consisting of histamine, liver-expressed chemokine (“LEC”)/CCL16, 4-methylhistamine, VUF-8430 (2-[(Aminoiminomethyl)amino]ethyl carbamimidothioic acid ester), OUP-16, and combinations thereof.

4. The transdermal drug formulation according to claim 1 further comprising:
a histamine type 1 receptor (“H1R”) agonist.

5. The transdermal drug formulation according to claim 4, wherein the H4R agonist and the H1R agonist are a single agent.

6. The transdermal drug formulation according to claim 1 further comprising:
an H1R agonist, a second H4R agonist, an agent that transiently disrupts claudin-1, -4, and/or -23 function within tight junctions, or a combination thereof.

7. The transdermal drug formulation according to claim 4 or 6, wherein the H1R agonist is selected from the group consisting of histamine, 2-Methylhistamine, 2-(3-Bromophenyl)histamine, Histaprodifen, 8R-Lisuride, 2-(2-thiazolyl)ethylamine (TEA), 2-pyridylethylamine (PEA), PEA dihydrochloride, and combinations thereof.

8. The transdermal drug formulation according to claim 1, wherein the transdermal drug formulation does not include histamine.

9. The transdermal drug formulation according to claim 1, wherein the transdermal drug formulation does not include a histamine type 2 receptor (“H2R”) agonist.

10. The transdermal drug formulation according to claim 1, wherein the carrier is selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

11.-14. (canceled)

15. An artificial vesicle comprising the drug formulation according to claim 1.

16. (canceled)

17. A transdermal vaccine formulation comprising:
a pharmaceutically suitable carrier;
an effective amount of an antigen or antigen-encoding nucleic acid molecule present in the carrier, and optionally one or more adjuvants; and
an H4R agonist.

18.-42. (canceled)

43. A method of administering a transdermal drug formulation to a subject comprising:
applying the transdermal drug formulation of claim 1 to an epithelial site on the subject.

44. A method of administering a transdermal vaccine formulation to a subject comprising:
applying the transdermal vaccine formulation of claim 17 to an epithelial site on the subject.

45. A method of disrupting an epithelial barrier comprising:
administering to an epithelial site an amount of an H4R agonist that transiently disrupts tight junctions, thereby disrupting barrier formation at the epithelial site.

46. A method of enhancing epithelial barrier formation in a patient, the method comprising:
administering to the patient at a site of epithelial disruption an amount of a formulation that comprises an H4R antagonist, thereby enhancing barrier formation at the site.

47.-65. (canceled)

66. A method of promoting epithelial function in an individual having compromised or immature epithelial function comprising:
providing a formulation comprising H4R antagonist, an H1R antagonist, or a combination thereof that enhances tight junction formation between epithelial cells; and
administering the formulation to a region of epithelia on an individual having reduced epithelial function at the region, thereby enhancing tight junction formation between epithelial cells and promoting epithelial function in the individual.

67.-75. (canceled)

76. A method of inhibiting pathogen infection or local spread of infection in the epithelia comprising:
providing a formulation comprising an H4R antagonist, an H1R antagonist, or a combination thereof; and
applying to a region of epithelia on an individual that is susceptible to pathogen infection an amount of the formulation that is effective to enhance epithelial barrier formation at the application site, thereby rendering the application site less susceptible to pathogen infection or local spread of infection.

77.-89. (canceled)

90. A method for temporarily inducing tight junction disruption, comprising using histamine and H4R agonist.

91.-92. (canceled)

93. A method for repairing an epithelial barrier, comprising using H4R antagonists (or other potential therapeutic targets downstream of H4R) in human disease mediated by histamine or another amine or another H4R ligand.
